(19) United States
(12) Patent Application Publication
(10) Pub. No.: US 2006/0025814 A1
(43) Pub. Date: Feb. 2, 2006

(54) Medical device

(75) Inventor: Tsuruo Hatori, Sagamihara-shi (JP)

Correspondence Address: Thomas Spinelli;Scully, Scott, Murphy & Presser, 400 Garden City Plaza, Garden City, NY, 11530, US

(73) Assignee: Olympus Corporation, Tokyo (JP)

(21) Appl. No.: 11/194,840

(22) Filed: Aug. 1, 2005

(30) Foreign Application Priority Data
Aug. 2, 2004 (JP) 2004-226007

(51) Int. Cl. A61B 17/28

(52) U.S. Cl. 606/205

(57) ABSTRACT

A medical device includes a portion which is expected to be subjected to an irreversible structural change due to an air pressure difference when the medical device is placed in a negative pressure in a sterilizing process. By the irreversible change of the aforementioned portion, an alarm display is exposed, or at least part of a function of the device is restricted or use of at least part of the function of the device is disabled.

### CROSS-REFERENCE TO RELATED APPLICATION

This application is based upon and claims the benefit of priority from prior Japanese Patent Application No. 2004-226007, filed Aug. 2, 2004, the entire contents of which is incorporated herein by reference.

### BACKGROUND OF THE INVENTION

1. Field of the Invention

The present invention relates to a medical device. More specifically, the present invention relates to preventing reuse of a single use medical device.

2. Description of the Related Art

In recent years, disposable medical devices, or single use medical devices (Single use Devices=SUD) are being used in the medical field. The disposable medical device or the single use medical device (hereinafter, referred simply as SUD medical device) can prevent infection diseases or the like by being discarded after each use for a treatment and replaced with a new SUD medical device for each treatment. Durability such as strength and required quality such as performance of the SUD medical devices are set to certain degrees on the premise of a single use.

While reusable medical devices are reused after being washed, disinfected, and sterilized to prevent cross infection among patients, the SUD medical devices are not manufactured on the assumption of reuse. Therefore, reuse of the SUD medical devices must be avoided.

Medical devices for preventing reuse of the SUD medical devices after a single use are proposed, for example, in Japanese Patent No. 2730990, JP-UM-A-3-101908, and JP-B-7-79839. In these patent publications, a medical introduction tube for introducing an instrument such as a catheter into a body cavity or a plug member provided at a pipe sleeve of an instrument insertion channel provided on an endoscope are disclosed. The plug member is provided to prevent blood or sewage in the body cavity from flowing back through the pipe sleeve of the medical introduction tube or the instrument insertion channel of the endoscope. The plug member includes a finger hook portion for hooking a finger when removing the plug member after being mounted to the pipe sleeve and used, and a breakable portion for causing the plug member to be broken from the finger hook portion when the finger hook portion is pulled, so as to be broken at the breakable portion when the finger hook portion is pulled to prevent reuse. In this manner, part of the plug member is adapted to be broken irreversibly to prevent the plug member from again being mounted to the pipe sleeve.

In JP-A-5-38324, a disposable type endoscope is provided with a pressure breakable portion (thinned portion), which is adapted to be broken when a pressure difference between the inside and the outside exceeds a predetermined pressure at an insertion portion of the endoscope. This device is adapted to be broken partly at the insertion portion when it cannot sustain the pressure difference between the inside and the outside of the endoscope stored in a chamber utilizing a property that inside the chamber is brought into a negative pressure at the time of ethylene oxide gas sterilization (referred to simply as EOG sterilization hereinafter), thereby preventing reuse of the device.

### BRIEF SUMMARY OF THE INVENTION

Accordingly, it is an object of the invention to provide a versatile reuse prevention technology in disposable medical devices.

A medical device according to the invention is provided with a portion which is expected to be subjected to irreversible structural change due to a pressure difference when the medical device is placed under a negative pressure in a process of sterilization. The device can be adapted to display a warning, or to restrict or disable at least part of the function thereof when such an irreversible change occurs at the aforementioned portion.

As the part which is expected to be subjected to the irreversible structural change due to the pressure difference, for example, an air chamber may be provided. The air chamber can be specifically used for this purpose only, without having other intended uses. In this arrangement, part of the medical device other than the air chamber can be manufactured with a required strength. When the air chamber has other intended uses, the medical device may easily be fragile at a position of the air chamber.

For example, the air chamber may include a recess which has an indication of “Do not use” on an interior surface thereof and a partitioning wall which seals the interior of the recess at the same pressure as the ambient pressure, and is adapted to be separated (or broken) when a difference between the ambient pressure and the atmospheric pressure around the medical device is greater than a predetermined value, such as when the atmospheric pressure becomes negative.

The recess may be provided at a part of a member which constitutes the medical device, or on a tag member of the medical device. The partitioning wall may be opaque. The opaque partitioning wall is advantageous since the indication of “Do not use” is not visible until the partitioning wall is separated or broken.

The part which is expected to be subjected to the irreversible structural change due to the pressure difference may be configured of a notch for causing an operating unit to be broken when a force exceeding a predetermined magnitude is applied to the operating unit, and a cover member mounted to a peripheral edge of the notch for preventing breakage of the operating unit from the notch and adapted to be separated when a difference between the ambient pressure and the atmospheric pressure around the medical device is greater than a predetermined value, such as when the atmospheric pressure becomes negative.

Furthermore, the part which is expected to be subjected to the irreversible structural change due to the pressure difference may be configured of a function restricting module provided on the operating unit of the medical device held and operated by an operator, so that an irreversible operation is achieved by an action of the pressure when a difference between the ambient pressure and the atmospheric pressure around the medical device is greater than a predetermined value, such as when the atmospheric pressure becomes negative. When the pressure difference is greater than the predetermined value, the function restricting module acts to restrict an operative function of the operating unit.

### BRIEF DESCRIPTION OF THE SEVERAL VIEWS OF THE DRAWINGS

These and other features, aspects, and advantages of the apparatus and methods of the present invention will become better understood with regard to the following description, appended claims, and accompanying drawings where:

FIG. 1 is a plan view showing a general structure of a disposable medial device according to a first embodiment;

FIG. 2 is an explanatory cross-sectional view of a recess and a partitioning wall provided on an operating unit of the disposable medical device as taken along section line 2-2 in FIG. 1;

FIG. 3 is a cross-sectional view showing a state in which the partitioning wall of FIG. 2 is separated from the recess provided on the operating unit of the disposable medical device;

FIG. 4 is an explanatory drawing showing an indication that reuse is prohibited provided in the recess of the disposable medical device;

FIG. 5 is an explanatory drawing showing another application of the disposable medical device according to the first embodiment;

FIG. 6 is a perspective view showing a general structure of a disposable medical device according to a second embodiment;

FIG. 7 is a cross-sectional view showing a structure of a holding portion of the operating unit of the disposable medical device of FIG. 6;

FIG. 8 is a plan view showing an action of the operating unit of FIG. 7;

FIG. 9 is a cross-sectional view showing a structure of the function restricting module to be used in a disposable medical device according to a third embodiment;

FIG. 10 is a cross-sectional view showing an operating state of the function restricting module of FIG. 9;

FIG. 11 is an explanatory drawing explaining an example in which the function restricting module of FIG. 9 is used in holding forceps;

FIG. 12 is an explanatory drawing showing an example in which the function restricting module of FIG. 9 is used in a trocar; and

FIG. 13 is an explanatory drawing showing an example in which the function restricting module of FIG. 9 is used in displacement forceps.

### DETAILED DESCRIPTION OF THE PREFERRED EMBODIMENTS

Preferred embodiments are described below with reference to the accompanying drawings.

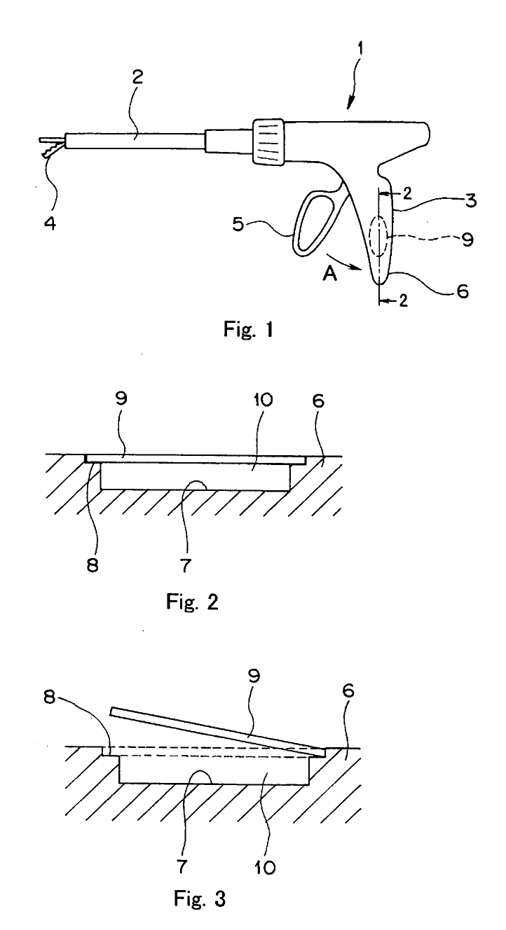

Referring now to FIG. 1 to FIG. 5, a medical device according to a first embodiment will be described.

In the first embodiment, holding forceps used in an endoscopic operation, which is a surgical operation conducted for an affected part while observing inside of the body cavity by an endoscope, will be described as an example of the medical device. The holding forceps 1 includes an insertion portion 2 and an operating unit 3 as shown in FIG. 1. The insertion portion 2 is inserted into a body cavity via a trocar, not shown, and a holding member 4 is provided at an extremity of the insertion portion 2. The holding member 4 is used for holding tissue of an affected part in the body cavity, and is remotely opened and closed using a holding wire, not shown, disposed in the insertion portion 2. The operating unit 3 includes a holding portion 6 formed into a shape like a gun grip and gripped by a hand of an operator, and a handle 5 operated by a finger of the operator who grips the holding portion 6. The handle 5 includes a holding wire connected thereto for operating the holding member 4, so that the holding member 4 opens or closes by moving the handle 5 in the direction indicated by Arrow A in FIG. 1.

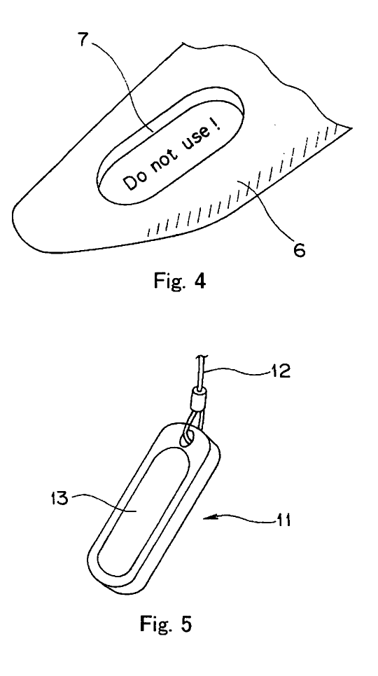

A recess 7 covered by a partitioning wall member 9 is provided on one side of the holding portion 6 of the operating unit 3 of the holding forceps 1. The recess 7 covered by the partitioning wall member 9 is formed to have a predetermined depth from a surface of the holding portion 6 as shown in FIG. 2. A peripheral edge portion 8 to which the periphery of the partitioning wall member 9 is fixed by an adhesive agent is formed on the peripheral edge of the recess 7. The recess 7 can have any shape, such as a circular, oval or rectangular shape. As shown in FIG. 4, the recess includes a reuse prohibiting indication such as “Do not reuse!” as shown in FIG. 4 or “Reuse is prohibited” which is not shown in the drawings.

An air chamber 10 is defined by the partitioning wall member 9 fixed to the peripheral edge portion 8 of the recess 7 by an adhesive agent, for example a silicon adhesive agent. In other words, when manufacturing the holding forceps 1, a sterilizing process such as gamma ray sterilization or electron beam sterilization of SUD medical devices is conducted under an atmospheric pressure and, in the process of the sterilization, the partitioning wall member 9 is adhered and fixed to the peripheral edge portion 8 of the recess 7 to seal sterilized air at an ambient pressure in the air chamber 10 inside the recess 7.

The partitioning wall member 9 is adhered and fixed so as to prevent the air in the air chamber 10 in the recess 7 from leaking out under an ambient pressure, and be separated easily when an atmospheric air pressure outside the air chamber 10 becomes significantly less than a pressure inside the air chamber 10, such as when the atmospheric pressure becomes negative, indicating that the holding forceps 1 is being sterilized (which is prohibited).

In this manner, the holding forceps 1 sterilized by sterilization process during manufacture of the SUD medical devices is sealed in a sterilized bag and presented to a medical institution.

In the medical institution, when conducting surgical treatment of an affected part using the holding forceps 1 while observing the affected part by the endoscope, the holding forceps 1 is taken out from the sterilized bag for use in a predetermined treatment. After having completed the treatment, in order to sterilize the holding forceps 1 in a sterilizing apparatus for ethylene oxide gas sterilization (EOG sterilization), autoclave sterilization, or hydrogen peroxide low-temperature plasma sterilization, which are popular sterilizing methods, the holding forceps 1 is stored in a sterilizing chamber and the sterilizing chamber is evacuated to produce a vacuum. At this time, the atmospheric pressure around the holding forceps 1 becomes negative with respect to the air pressure in the air chamber 10 in the recess 7, and hence a difference in air pressure results between the inside and the outside of the air chamber 10. When the difference in air pressure exceeds a predetermined value, the partitioning wall member 9 adhered and fixed to the peripheral edge portion 8 of the recess 7 is separated as shown in FIG. 3, and the do-not-reuse indication displayed on the bottom surface of the recess 7 is exposed and visible. Since the do-not-reuse indication is exposed when the partitioning wall member 9 is separated, the operator can verify the fact that the holding forceps 1, with the partitioning wall member 9 separated, is an SUD medial device and that the sterilizing process for reuse has been conducted for the holding forceps 1 (which is a SUD medical device in which sterilization is prohibited). Therefore, the operator can easily judge whether the holding forceps 1 is reusable or not.

The recess 7 covered by the partitioning wall member 9 and used for the do-no-reuse indication may be any shape and size which enables visual recognition of the do-not-reuse indication. Furthermore, the recess 7 can be provided at any position on the operating unit 3. Therefore, a versatile do-not-reuse indication can be employed in many types of SUD medical devices.

Although the holding forceps 1 are described above as an example, the invention can be also applied to various treatment forceps such as an incision forceps, e.g. an electric surgical knife or a high-frequency knife for incising viscous membranes other than the holding forceps.

As another application of the first embodiment, as shown in FIG. 5, a tag 11 to be attached to the SUD medical device can be employed. The tag 11 includes a connecting member 12 to be connected and fixed to the SUD medical device, and a recess (not shown) covered by a partitioning wall member 13 provided on one side of the tag 11. The recess covered by the partitioning wall member 13 of the tag 11 has the same structure as the recess 7 described above, and the partitioning wall member 13 is the same as the partitioning wall member 9 described above.

Since the tag 11 can be attached easily to various SUD medial devices, when the atmospheric pressure in the chamber becomes negative when being stored in the chamber and conducting the sterilizing process, the partitioning wall member 13 of the tag 11 is separated and hence the do-not-reuse display provided on the recess is exposed and visible, whereby recognition of the fact that the device is a sterilized SUD medical device and verification of being sterilized are enabled.

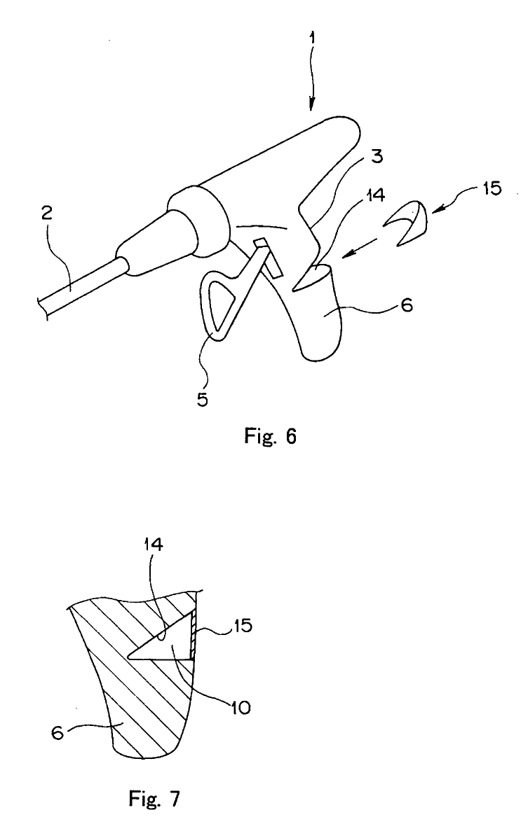

Subsequently, referring to FIG. 6 to FIG. 8, the medical device according to a second embodiment will be described. The same parts as the medical device described based on FIG. 1 and FIG. 2 are represented by the same reference numerals and detailed description thereof is omitted.

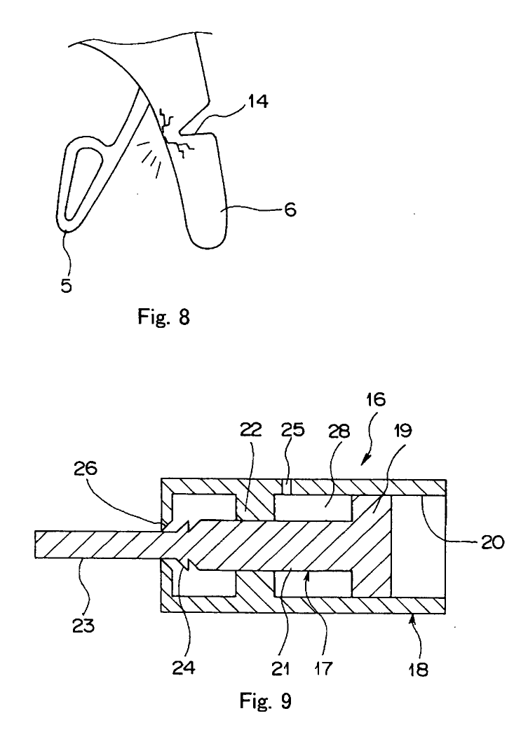

The holding portion 6, which is held by the operator, of the operating unit of the holding forceps 1 as the SUD medical device according to the second embodiment is formed with a notch 14 cut into a V-shape opening from a proximal side toward a distal side, which is opposite from the handle 5, as shown in FIG. 6. The notch 14 has a depth such that when the operator holds the holding portion 6 and operates the handle 5 toward the holding portion 6, the holding portion 6 is broken by the operating force from the notch 14 as shown in FIG. 8.

A cover member 15 is adhered and fixed to the peripheral edge of the opening of the notch 14 so as to cover the notch 14. The cover member 15 is a reinforcing member adhered and fixed to the peripheral edge of the opening of the notch 14 for preventing the holding portion 6 from being broken by a force exerted to the holding portion 6 when the handle 5 is operated. An air chamber 10 is defined by the notch 14 and the cover member 15.

Adhesion and fixation of the cover member 15 to the peripheral edge of the opening of the notch 14 is performed under the ambient pressure as in the case of the first embodiment described above. The cover member 15 is adhered and fixed to reinforce the holding portion 6 so as not to be broken from the notch 14 and to prevent leakage of air from the air chamber 10 toward the outside. The cover member 15 is adhered and fixed so as to be separated easily when the outside air pressure becomes negative, thus exceeding a predetermined magnitude with respect to a difference between the pressure in the air chamber 10 and the outside pressure.

When the holding forceps 1 is stored in a chamber for sterilization for performing, for example, EOG sterilization and bringing the inside of the chamber into a negative pressure after having used the holding forceps 1 as the SUD medical device in this configuration under the endoscopic observation by the operator, a difference in pressure between the internal air pressure in the air chamber 10 and the external atmospheric pressure is resulted, and the cover member 15 is separated from the peripheral edge of the opening of the notch 14 when such pressure difference exceeds a predetermined value.

In the holding forceps 1 with the cover member 15 separated from the notch 14, when the handle 5 of the operating unit 3 is operated, the operating force generates a moment which bends the bottom of the holding portion 6 toward the handle 5 from the notch 14 of the holding portion 6. Consequently, as shown in FIG. 8, the holding portion 6 is broken. According to the second embodiment, since the holding portion 6 is broken, the holding forceps 1 cannot be reused, and hence reuse of the SUD medial device is prevented in advance of a reuse.

Referring now to FIG. 9 to FIG. 13, a medical device according to a third embodiment will be described.

A function restricting module 16 used in a SUD device as a medical device according to a third embodiment restricts reuse of the SUD medical device by restricting or disabling at least part of the function of the medical device. The function restricting module 16 includes a slider member 17 and a cylindrical member 18 as shown in FIG. 9. The slider member 17 includes a piston portion 19, a shaft portion 21, a pin portion 23 and a male engaging portion 24. The piston portion 19 is provided at a first end of the slider member 17, and formed into a disk shape. The shaft portion 21 extends by a predetermined dimension from the axial center of the piston portion 19 to another end, and has a column shape having an outer diameter smaller than the piston portion 19. The pin portion 23 extends axially by a predetermined dimension towards the other end of the shaft portion 21, and has a column shape having an outer diameter smaller than the shaft portion 21. The male engaging portion 24 is provided on the outer periphery at a boundary position between the pin portion 23 and the shaft portion 21.

The cylindrical member 18 includes a cylinder portion 20, a supporting portion 22, and a female engaging portion 26. An outer periphery of the piston portion 19 of the slider member 17 is fitted into the cylinder portion 20, and the piston portion 19 slides therein. The supporting portion 22 is formed on the central portion of the inner periphery of the cylinder portion 20 and the outer periphery of the shaft portion 21 of the slider member is fitted into the supporting portion 22 and slides therein. The female engaging portion 26 is provided in the radial direction at the other end of the cylinder portion 20 with respect to the supporting portion 22, and the pin portion 23 of the slider member 17 is fitted and slides therein, and is provided on the outer periphery around a boundary between the pin portion 23 and the shaft portion 21.

In other words, when a distal end of the pin portion 23 of the slider member 17 is inserted through the cylinder portion 20, the supporting portion 22 and the female engaging portion 26, the piston portion 19 is slidably fitted to the internal periphery of the cylinder portion 20, the shaft portion 21 is slidably fitted to the supporting portion 22, and the pin portion 23 is slidably fitted to the female engaging portion 26 respectively.

The piston portion 19 of the slider member 17 is slidably fitted into the cylinder portion 20 of the cylindrical member 18, and a space defined between the piston portion 19 and the supporting portion 22 is an air chamber 28. In order to communicate the air chamber 28 defined by the piston portion 19 and the supporting portion 22 with the outside air, an air ventilation hole 25 is formed through a portion near the supporting portion 22.

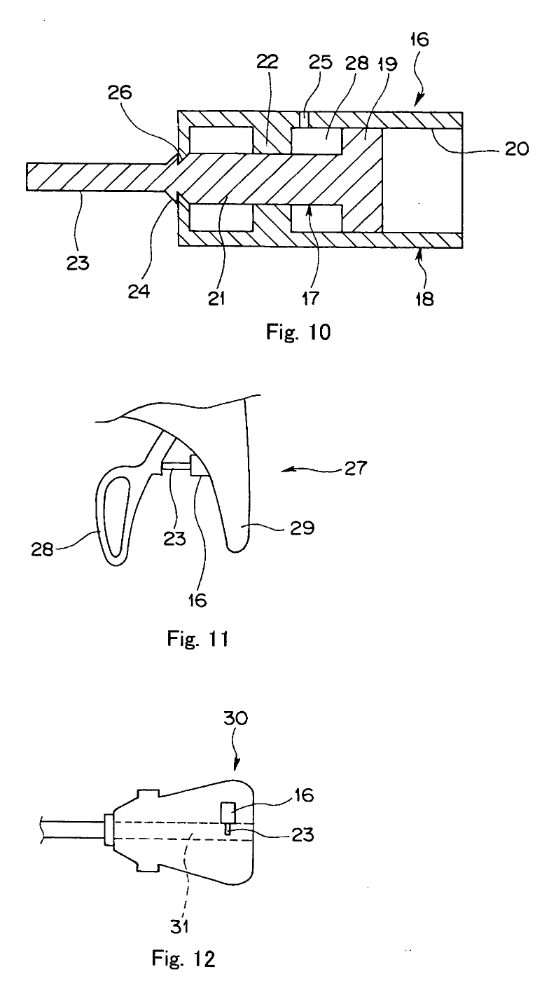

As shown in FIG. 9, in the function restricting module 16 in this arrangement, the piston portion 19 of the slider member 17 is fitted to the inner periphery of the cylinder portion 20, the shaft portion 21 of the slider member 17 is fitted to the supporting portion 22 of the cylindrical member 18, the pin portion 23 of the slider member 17 is fitted to the female engaging portion 26 of the cylindrical member 18, whereby the air chamber 28 is defined by the piston portion 19, the cylinder portion 20 and the supporting portion 22. In a state in which the male engaging portion 24 provided on the shaft portion 21 is positioned between the supporting portion 22 and the female engaging portion 26. It is assumed that the SUD medical device having the function restricting module of this embodiment is stored in the sterilizing chamber for sterilization and the atmospheric pressure of the cylindrical member 18 becomes negative. Then, the atmosphere of the air chamber 28 is evacuated (the pressure inside of the sterilization chamber decreases) through the air-ventilation hole 25. Therefore, as shown in FIG. 10, the piston portion 19, the shaft portion 21, and the pin portion 23 slide distally (i.e., toward the left in FIG. 10). Consequently, the male engaging portion 24 of the shaft portion 21 engages and locks with the female engaging portion 26 on the cylindrical member 18. In other words, the slider member 17 slidably inserted into the cylindrical member 18 is slid by the atmospheric pressure and hence the male engaging portion 24 provided on the shaft portion 21 engages with the female engaging portion 26, whereby the pin portion 23 of the slider member 17 is axially projected from the cylindrical member 18 and locked at a predetermined position. By the engagement and locking between the male engaging portion 24 and the female engaging portion 26, the slider member 17 cannot be restored to its original position even when the atmospheric pressure changes.

The function restricting module 16 is stored and disposed in a holding portion 29 of a holding forceps 27, which is the same as the holding forceps 1 as described above in the first and second embodiments as shown in FIG. 11 so that the pin portion 23 projects toward the handle 28 side. In other words, at the time of manufacture of the holding forceps 27 as the SUD medical device, the function restricting module 16 in a state shown in FIG. 9 is disposed in the holding portion 29 of the holding forceps 27. When the handle 28 of the holding forceps 27 provided with the function restricting module 16 in this state is operated, the holding portion provided at the distal end of the insertion portion, not shown, of the holding forceps 27 can be opened and closed.

When the operator performs the treatment using the holding forceps 27 provided with the function restricting module 16, and the holding forceps 27 are stored in the sterilizing chamber, for example, for EOG sterilization to brings the inside of the chamber into a negative pressure, a differential pressure is resulted between the internal air pressure in the air chamber 28 of the function restricting module 16 stored and disposed in the holding portion 29 and the atmospheric pressure on the outside, and the slider member 17 slides to the position shown in FIG. 10. Then, the pin portion 23 projects toward the handle 28, and the male engaging portion 24 and the female engaging portion 26 are engaged and locked. Accordingly, if the operator operates the handle 28 of the holding forceps 27 after the EOG sterilization, the handle 28 abuts against the pin portion 23 and hence the operation of the handle 28 is disabled and reuse for another treatment is prevented. The cylinder portion or function thereof can also be integrally formed with the holding portion 29 or other portion of the holding forceps 27.

Subsequently, an example in which the function restricting module 16 is used in a trocar 30 as shown in FIG. 12 will be described. The function restricting module 16 is stored and disposed in the cylindrical member 31 in which an inner needle (not shown) of the trocar 30 is inserted in the orthogonal direction. In other words, at the time of manufacturing the trocar 30, which is the SUD medical device, the function restricting module 16 is stored and disposed in a state in which a distal end of the pin portion 23 is directed to the cylindrical member 31 of the trocar 30 as shown in FIG. 9. The inner needle is inserted to the trocar 30 provided with the function restricting module 16 in this state from the cylindrical member 31, and then the treatment is performed.

After having finished the treatment using the trocar 30 provided with the function restricting module 16 by the operator, if the trocar 30 is stored in the chamber for EOG sterilization and the inside of the chamber is brought into a negative pressure, a difference results between the internal air pressure in the air chamber 28 of the function preventing module 16 stored and disposed in the trocar 30 and the atmospheric pressure on the outside, and the slider portion 17 slides to the position shown in FIG. 10. Then, the pin portion 23 projects into the cylindrical member 31, and the male engaging portion 24 and the female engaging portion 26 are engaged and locked. Accordingly, if the operator inserts the inner needle or other instrument into the cylindrical member 31 of the trocar 30 after the trocar 30 is subjected to EOG sterilization, the inner needle abuts the pin portion 23, and hence insertion of the inner needle is disabled, whereby reuse for another treatment is prevented.

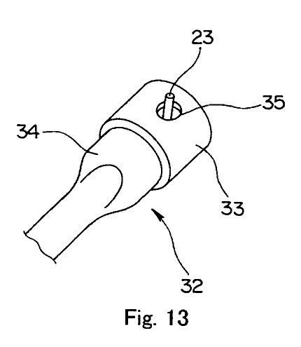

In addition, a case in which the function restricting module 16 is used in displacement forceps 32 as shown in FIG. 13 will be described. The operating unit 34 at the proximal end on the operator's side is provided with a dial 33 for opening and closing the distal end of forceps (not shown) for holding an internal organ in the body cavity. In other words, it is adapted to be capable of adjusting the opening and closing angle of the distal end of the forceps when rotating the dial 33. A hole 35 is provided on part of the dial 33 of the operating unit 34 of the displacement forceps 32. In a state in which the distal end of the forceps is brought into a closed state by rotating the dial 33, the function restricting module 16 in a state shown in FIG. 9 is stored in the operating unit 34 so that the pin portion 23 of the function restricting module 16 is positioned immediately below the hole 35 provided on the dial 33.

If the operator stores the displacement forceps 32 provided with the function restricting module 16 in the sterilizing chamber for performing for example, EOG sterilization after having completed the treatment using the displacement forceps 32, and the chamber pressure becomes negative, a difference between the internal air pressure in the air chamber 28 of the function restricting module 16 stored and disposed in the operating unit 34 of the displacement forceps 32 and the external atmospheric pressure results, and the slider portion 17 slides to the position shown in FIG. 10. Then, the pin portion 23 projects into the hole 35 provided on the dial 33, and the male engaging portion 24 and the female engaging portion 26 are engaged and locked. Accordingly, the pin portion 23 projects from the hole 35 on the dial 33 of the displacement forceps 32 after the EOG sterilization 32, the dial 33 can no longer be rotated, whereby reuse for another treatment is prevented.

In this manner, by storing and arranging the function restricting module 16 at a position where the operation of the main function of the SUD medical device can be restricted, the SUD medical device can be brought into a state in which reuse is restricted. Therefore, the function restricting module 16 can be applied to a plurality of types of SUD medical devices and is superior in versatility.

While there has been shown and described what are considered to be preferred embodiments of the invention, it will, of course, be understood that various modifications and changes in form or detail could readily be made without departing from the spirit of the invention. It is therefore intended that the invention not be limited to the exact forms described and illustrated, but constructed to cover all modifications that may fall within the scope of the appended claims.

What is claimed is:

1. A medical device comprising:
portions defining a recess, the recess having an indication prohibiting use of the medical device on an inner surface thereof; and
a partitioning wall member which seals an interior of the recess at a first air pressure and is adapted to be separated or broken to expose the indication when a difference between the first air pressure in the recess and an atmospheric pressure around the medical device is above a predetermined value.

2. The medical device according to claim 1, wherein the recess is provided on a member which constitutes the medical device.

3. The medical device according to claim 1, wherein the recess is provided on a tag member connected to the medical device.

4. The medical device according to claim 1, wherein the partitioning wall member is opaque.

5. The medical device according to claim 1, wherein the atmospheric pressure is negative.

6. A medical device comprising:
an operating unit having a notch provided so that the operating unit is broken when a force exceeding a predetermined magnitude is exerted to the operating unit; and
a cover member mounted to a peripheral edge of the notch so as to cover the notch for preventing the notch from breaking, the notch and cover member defining an air chamber, the cover member being adapted to be separated or broken when a difference between a pressure inside the air chamber and an atmospheric pressure around the medical device is above a predetermined value.

7. The medical device according to claim 6, wherein the atmospheric pressure is negative.

8. A medical device comprising:
an operating unit to be held and operated by an operator; and
a function restricting module provided on the operating unit for irreversibly restricting an operating function of the operating unit when an atmospheric pressure around the medical device becomes negative.

9. The medical device according to claim 8, wherein the function restricting module comprises:
a cylindrical member having a cylinder portion;
a slider member having a piston portion to be fitted into the cylinder portion; and
an engaging portion for irreversibly engaging the slider member when a pressure inside an air chamber defined by the cylinder portion and the piston portion becomes negative thereby causing the slider portion to slide together with the piston portion by a predetermined distance, and operation of the operating unit of the medical device is disabled by a distal end of the slider portion.

10. A single use medical device comprising:
a partitioning wall defining at least a portion of an air chamber, the partitioning wall being adapted to be broken or separated due to a predetermined difference between a pressure outside the air chamber and a pressure inside the air chamber; and
an alarm display portion which is exposed when the partitioning wall is broken or separated.

11. A single use medical device comprising a module for irreversibly restricting a function of the single use medical device due to a pressure action when an atmospheric pressure around the single use medical device is lowered a predetermined amount.

12. A method of preventing reuse of a single use medical device, the method comprising:
causing a irreversible change in structure in an air chamber provided on the single use medical device by a pressure difference when the single use medical device is stored in a sterilizing chamber and placed in a negative pressure; and
causing at least one of an alarm display to be exposed and restricting or disabling use of at least part of a function of the single use medical device by the irreversible change.

13. The method according to claim 12, wherein the irreversible change in structure comprises opening a cover member to expose the alarm display.

14. The method according to claim 12, wherein the irreversible change in structure comprises extending a member to interfere with an operation of the single use medical device.
